(19) United States
(12) Patent Application Publication
(10) Pub. No.: US 2013/0053939 A1
(43) Pub. Date: Feb. 28, 2013

(54) METHOD FOR MANUFACTURING CYLINDRICAL NITINOL OBJECTS FROM SHEET

(71) Applicants: Tom Duerig, Fremont, CA (US); Christine Trepanier, Union City, CA (US); Lot Vien, San Jose, CA (US); Dieter Stoeckel, Carmel Valley, CA (US); Payman Saffari, San Jose, CA (US)

(72) Inventors: Tom Duerig, Fremont, CA (US); Christine Trepanier, Union City, CA (US); Lot Vien, San Jose, CA (US); Dieter Stoeckel, Carmel Valley, CA (US); Payman Saffari, San Jose, CA (US)

(21) Appl. No.: 13/600,149

(22) Filed: Aug. 30, 2012

Related U.S. Application Data
(60) Provisional application No. 61/529,158, filed on Aug. 30, 2011.

(51) Int. Cl. C21D 8/00; B21D 51/10; A61F 2/82; B21D 31/00

(52) U.S. Cl. 623/1.1; 148/559; 72/379.2; 428/544

(57) ABSTRACT

An article cut from a metallic sheet, has a first pattern of struts forming a plurality of inner apexes situated substantially within a plane that contains the plurality of the inner apexes. There is a second pattern of struts forming a plurality of outer apexes that are situated substantially within the plane containing the inner apexes. Each outer apex has at least one strut in common with an adjacent inner apex. There is also described a method of forming a non-planar three dimensional structure by patterning a planar sheet of material to form an article having a plurality of inner apexes, a plurality of outer apexes with a common curvilinear strut between an inner apex and an adjacent outer apex. Thereafter, everting the article into a non-planar three dimensional structure with the inner apexes at one end of the structure and the outer apexes at another end of the structure.

### CROSS REFERENCE TO RELATED APPLICATIONS

This application claims the benefit under 35 U.S.C. 119 of U.S. Provisional Patent Application No. 61/529,158, filed Aug. 30, 2011, titled “AN IMPROVED METHOD FOR MANUFACTURING CYLINDRICAL NITINOL OBJECTS FROM SHEET.” This application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates to techniques used in the fabrication of cylindrical articles from a flat sheet. More specifically, this application relates to improved manufacturing techniques for stents from a Nitinol sheet.

### BACKGROUND

Many medical devices are cut from tubing by either laser or Electro Discharge Machining (EDM), particularly Nitinol devices. Nitinol tubing, however, is very expensive, and there are various other drawbacks from making devices through such a process. It is also known, as described in U.S. Pat. No. 5,907,893 (hereinafter, “the '893 patent”), that one can cut certain patterns from flat sheet and then form the cut shape into the desired cylindrical shape; the disclosed method, however, results in shapes that are not ideal (being asymmetric from end-to-end).

FIGS. 1A and 1B reproduce FIGS. 18 and 19 of the '893 patent, representing the known art for forming a cylindrical stent from flat sheet. Many other examples are provided in the patent, but this transfiguration best represents the teachings of the existing art. More specifically, the '893 patent describes a procedure in which the planar eversible article 500 shape shown in FIG. 1A is cut from flat Nitinol sheet. The shape has an open star shaped pattern with a plurality of straight struts 502. The struts 502 are arranged as a plurality of circumferentially spaced-apart V-shaped components with adjacent V-shaped components meeting or joining at its radially outward end to produce inner apexes 510 and outer apexes 520 and included angles 525. Next, the article 500 is deformed by placing a cylindrical mandrel into that shape, thus inverting it into an approximate cylinder, setting that second shape by heat treating while on the deforming mandrel, then removing the mandrel, finally achieving a same shape as a conventionally manufactured stent as shown in FIG. 1B.

We believe that when one actually cuts the articles shape 500 shown in FIG. 1A, one does not easily obtain the shape of the article 500 shown in FIG. 1B. In fact, several complex manipulations are required in order to achieve the desired configuration shown in FIG. 1B. More specifically, when one everts the structure 500 shown in FIG. 1A by sliding it onto an approximately cylindrical mandrel, one finds that (1) the struts are not straight, but have an asymmetric curvature, with the included angles greater on one end than the other, (2) many of the struts 502 no longer rest in a cylindrical plane, but jut outwards, and (3) a taper results unless very high shape setting temperatures are used, which would result in an undesirable loss in strength. While the second and third observations are slightly problematic, the first is of paramount importance because the strains that result from subsequent diametral pulsing, or during crimping into a catheter are substantially greater than they would be if the two ends were the same.

While one can imagine complex shape setting processes that would result in end-to-end symmetry, specifically complex tooling that forces the struts into the desired position and that holds the struts in that position during heat treatment. Such tools are expensive and difficult to use, however, and significantly add to the cost of the product, since the primary goal of using sheet in the first place rather than tubing is cost reduction, such measures are not practical.

What is needed are improved techniques for pattern design and selection as well as cutting the pattern from sheet that, when formed into a cylindrical shape, forms a more symmetric finished shape. It is believed that improvements to the manufacturing of devices from sheet cut patterns will have substantially more uniform stresses and strains, thus providing improved durability compared to conventionally manufactured devices such as described in the '893.

### SUMMARY OF THE DISCLOSURE

In one aspect, there is an article cut from a metallic sheet having a first pattern of struts forming a plurality of inner apexes situated substantially within a plane that contains the plurality of the inner apexes. The struts used to form each inner apex are arranged to form outward opening angles. There is also a second pattern of struts forming a plurality of outer apexes that are situated substantially within the plane containing the inner apexes. The struts used to form each outer apex are arranged to form inward opening angles. Each outer apex has at least one strut in common with an adjacent inner apex. The struts forming the inward opening angles are convergent and the struts forming the outward opening angles are divergent. In one alternative, the first pattern of struts and the second pattern of struts form a continuous closed shape. In one embodiment, the closed shape is a star pattern, but may include other closed shapes. In still another aspect, the struts forming the inner apexes are curved in a portion of the strut immediately adjacent to the inner apex. In another aspect, each strut is generally curvilinear along its length between an outer apex and an inner apex.

In one alternative aspect, the two dimensional article is substantial planar. Still further, the first pattern and the second pattern are arranged to form an alternating arrangement of an inner apex adjacent to an outer apex. In one embodiment, the shape and size of the struts and apexes in the article are selected so that the article forms a non-planar article when everted. The non-planar article is substantially cylindrically symmetrical along the longitudinal axis of the non-planar article, in some embodiments. In other embodiments, after everting the article, the article forms a device having an approximate conical or cylindrical symmetry. In one specific embodiment, the non-planar article is a stent adapted and configured for the vasculature of a mammal.

In another alternative embodiment, there is a substantially planar article cut from a metallic sheet for eversion into a non-planar three dimensional structure. In this embodiment, the article includes a first pattern of struts forming a plurality of inner apexes and a second pattern of struts forming a plurality of outer apexes positioned in relation to the first pattern of struts to provide an alternating pattern of an inner apex and an adjacent outer apex. There is a common strut that joins the inner apex to the adjacent outer apex. A straight line distance between the inner apex and the adjacent outer apex is shorter than a path length along the common strut from the inner apex to the adjacent outer apex. After forming the non-planar three dimensional structure, the straight line distance between the inner apex and the adjacent outer apex is longer than in the substantially planar article. In one aspect, there is a curvature of the common strut is greater in the substantially planar article than after conducting an eversion process on the article. The plurality of inner apexes define a plurality of outward opening angles and the struts forming the outward opening angle. The outward opening angles may be convergent or divergent.

In another alternative embodiment, the plurality of outer apexes define a plurality of inward opening angles. In one aspect, the struts forming the inward opening angle are divergent, while in other embodiments the angles may be convergent. In one embodiment, the angle formed by an inner apex of the substantially planar article is greater than the angle formed by the same inner apex after performing an eversion process. In another embodiment, the angle formed by an outer apex of the substantially planar article is less than the angle formed by the same outer apex after performing an eversion process. In one aspect, each common strut has a first curved portion adjacent to an inner apex and a second different curved portion adjacent to an outer apex. In still other aspects, the non-planar three dimensional structure further comprising a cylindrical symmetry or a conical symmetry along the main longitudinal axis of the structure.

In another alternative embodiment, there is provides a method of forming a non-planar three dimensional structure by patterning a planar sheet of material. The pattern forms an article having a plurality of inner apexes, a plurality of outer apexes with a common curvilinear strut between an inner apex and an adjacent outer apex. The pattern also having inner apexes forming divergent wall included angles and the outer apexes forming converging wall included angles. Thereafter, everting the article into a non-planar three dimensional structure having the inner apexes at a first end of the structure and the outer apexes at a second end of the structure. In one aspect, the size of the inner apexes included angle is reduced after the everting step. In another aspect, the side of the outer apexes included angle is increased after the everting step. In still another embodiment, the method also includes performing a heat treatment operation to substantially remove a stress or a strain introduced into the article during the everting step. There may also be a step of reducing the diameter of the non-planar three dimensional structure or expanding the diameter of the structure. In one aspect the everting process also includes forming the non-planar three dimensional structure into having a substantially cylindrical symmetry along the main longitudinal axis of the structure. In another aspect, the everting process includes forming the non-planar three dimensional structure into having a substantially conical symmetry along the main longitudinal axis of the structure.

In still other alternative embodiments, there is a medical device made by cutting flat sheet into a shape wherein the struts are curvilinear, then inverting the overall shape to form a device that is has approximate conical or cylindrical symmetry. The curvilinear struts define outer apexes having larger included angles than the included angles formed by the interior apexes. In other alternatives of any of the above, each strut has a first curved portion adjacent to an inner apex and a second different curved portion adjacent to an outer apex. In any of the above described alternatives, the article or device is formed from Nitinol. Still further, in any of the above described alternatives or aspects the device is inverted by inserting a mandrel in the interior of the cut sheet and the Nitinol device is heated to the temperature range 250° C. to 600° C. to set the inverted shape. In some embodiments, a tapered mandrel is used to accommodate springback in the everted structure. In some aspects, an additional constraint is added to forcibly hold the inverted part against the mandrel. There is also disclosed a method of formed a device from a flat sheet of material including cutting an article from the flat sheet having a plurality of interconnected curvilinear struts forming a plurality of inner apexes and outer apexes. Thereafter, everting the article to form a device having symmetry along its main longitudinal axis. In one aspect, the symmetry along its main longitudinal axis comprising conical symmetry. In another aspect, the symmetry along its main longitudinal axis comprising cylindrical symmetry.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A (originally FIG. 18 of the '893 patent) illustrates a planar eversible structure having a continuous annular zig-zag pattern constructed in accordance with the manufacturing principles described in the '893 patent;

FIG. 1B (originally FIG. 19 of the '893 patent) illustrates the structure of FIG. 1A purportedly after it has been everted according to the methods described in the '893 patent;

FIG. 2A is a top down view of an actual pattern as cut from flat sheet according to the prior art methods described in '893 patent FIG. 18;

FIG. 2B is an isometric view of the patterned part photographed in FIG. 2A after forming onto a cylindrical mandrel. The top of the device illustrated in FIG. 2B corresponds to inside of the star in FIG. 2A, the bottom of the device (i.e., towards the bottom of the page) is the outside of the star in FIG. 2A.

FIG. 3A is a top down view of an optimized pattern as cut from flat sheet according to an embodiment of the present invention.

FIG. 3B is an isometric view of the shape of the part in FIG. 3A that results after forming onto a cylindrical mandrel. The top of the device in FIG. 3B corresponds to inside of the star in FIG. 3A, and the bottom of the device (towards the bottom of the page corresponds to the outside of the star in FIG. 3A.

FIGS. 4A-F compare the crimped strains of parts made per the prior art (FIGS. 4A, 4C, 4E) to those made according to an embodiment the present invention (FIGS. 4B, 4D, and 4F). FIGS. 4A and 4B compare an isometric view of the prior art initial geometry with an isometric view of an embodiment of the present invention initial geometry, respectively. FIGS. 4C and 4D show isometric views of both geometries of FIGS. 4A and 4B, respectively, as formed into a cylindrical shape of 18 mm diameter (performed by finite element analysis). FIGS. 4E and 4F are isometric views that compare the strains after crimping the shapes to 4 mm diameter. A comparison of FIGS. 4E and 4F reveals that the peak strains are reduced from 7.5% (FIG. 4E prior art) to 6.9% (FIG. 4F according to an embodiment of the invention).

FIG. 4G is an enlarged portion of one end of the embodiment shown in FIG. 4E.

FIG. 4H is an enlarged portion of another end of the embodiment shown in FIG. 4E.

FIG. 4I is an enlarged portion of one end of the embodiment shown in FIG. 4H.

FIG. 4J is an enlarged portion of another end of the embodiment shown in FIG. 4H.

FIGS. 5A and 5B are top down and isometric views, respectively, of a more complex example of a pattern with larger apex angles on the outside than on the inside, and the curvilinear strut configuration (FIG. 5A) and then after application of the eversion principle into the device in FIG. 5B.

FIGS. 6A and 6B illustrate another pattern and device as in FIG. 3, but this time set in a semi-conical shape, and with a cross section that is oval rather than circular as the pattern (FIG. 6A) and after the eversion principal, formed into the device shown in FIG. 6B.

### DETAILED DESCRIPTION

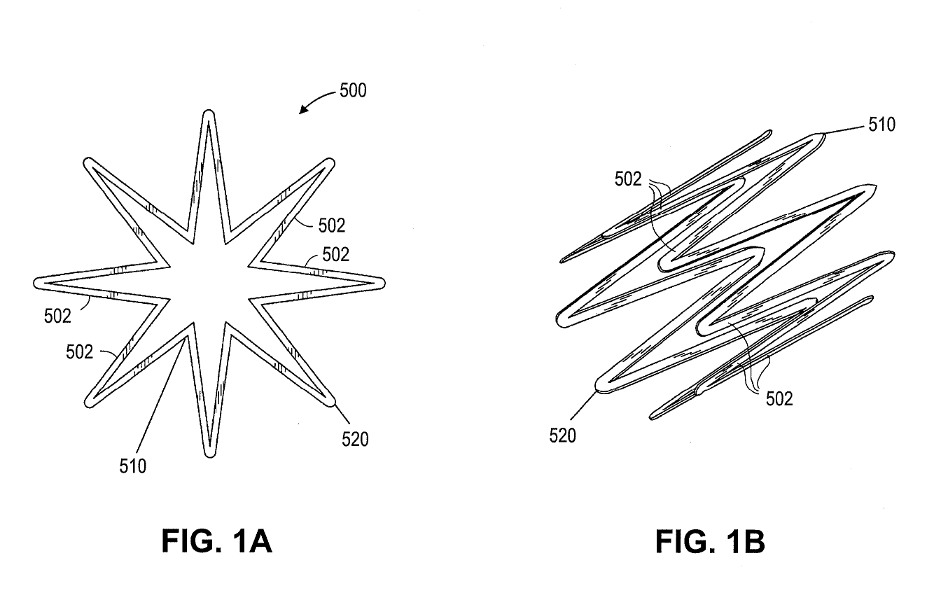

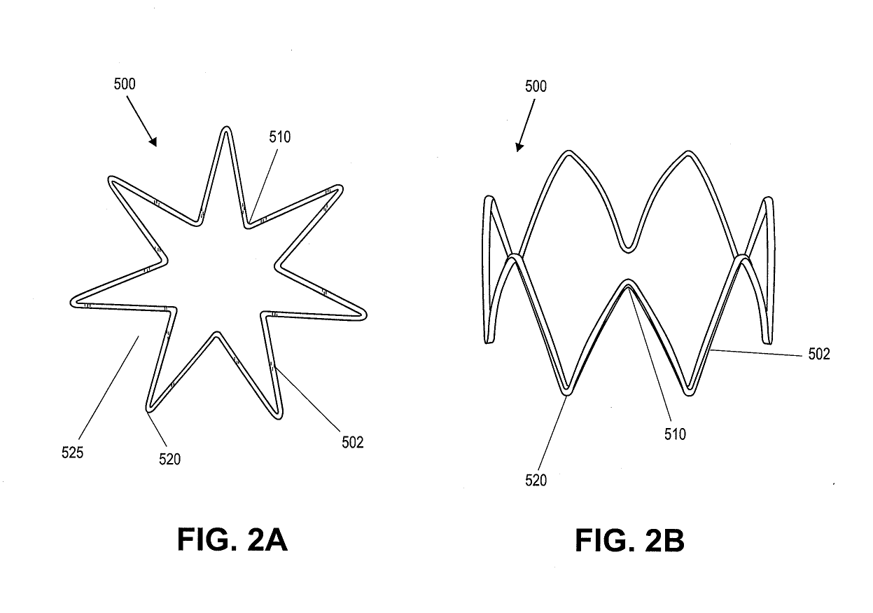

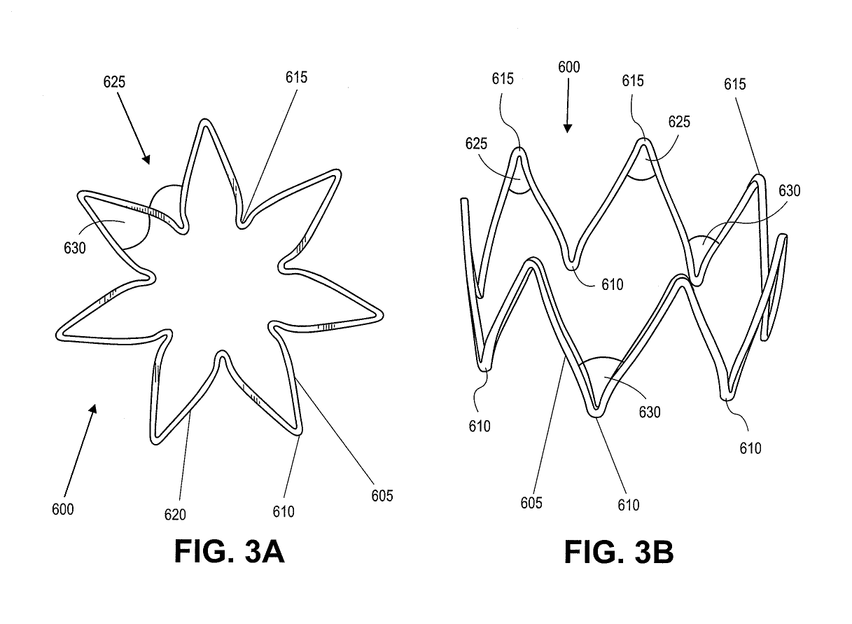

Aspects of the invention relate to ways to simplify manufacturing processes for devices formed from a sheet starting material. In addition, the devices formed according to the embodiments described herein achieve devices that—when everted into final form—maintain or achieve cylindrically symmetric devices. Furthermore, we have found that one solution to the primary problem identified above can be remedied if one cuts a different shape from the sheet than that shown in FIGS. 1A and 2A. Importantly, as set out in FIGS. 3, 5, and 6, this technique applies, as well, to the other examples shown in the prior art. Instead of the straight struts 502 described above, one instead cuts a flat pattern wherein the struts are curved, so that the included angles on the outside of the “star” are greater than shown in FIG. 2A, and in which the struts arch inwards.

Turning now to FIG. 3A that is a top down view of an eversible article 600. The article 600 is an example of an article cut from a metallic sheet. The article 600 includes struts 605 formed into inner apexes 615 and outer apexes 610. There is a first pattern of struts 605 forming a plurality of inner apexes 615 situated substantially within a plane that contains the plurality of the inner apexes. The struts 605 used to form each inner apex 615 are arranged to form outward opening angles 625. There is also a second pattern of struts 605 forming a plurality of outer apexes 610. The outer apexes are situated substantially within the plane containing the inner apexes 605. In other words, the patterned articles are substantially planar in that they are cut or formed from planar sheets. The struts 605 used to form each outer apex 610 are arranged to form inward opening angles 630. In this embodiment, the adjacent struts 605 are curved adjacent to the inner apex 615. The struts 605 are common struts between an outer apex 610 and an adjacent inner apex 615 and have a curvilinear shape in contrast the straight noticeably V-shaped struts and angles of FIGS. 1A and 1B.

Also illustrated in the embodiment of the article 600 in FIG. 3A is that each outer apex has at least one strut in common with an adjacent inner apex. The struts forming the inward opening angles are convergent and the struts forming the outward opening angles are divergent. In one alternative, the first pattern of struts and the second pattern of struts form a continuous closed shape. In one embodiment, the closed shape is a star pattern, but may include other closed shapes. In still another aspect, the struts forming the inner apexes are curved in a portion 620 of the strut immediately adjacent to the inner apex. In another aspect, each strut is generally curvilinear along its length between an outer apex and an inner apex.

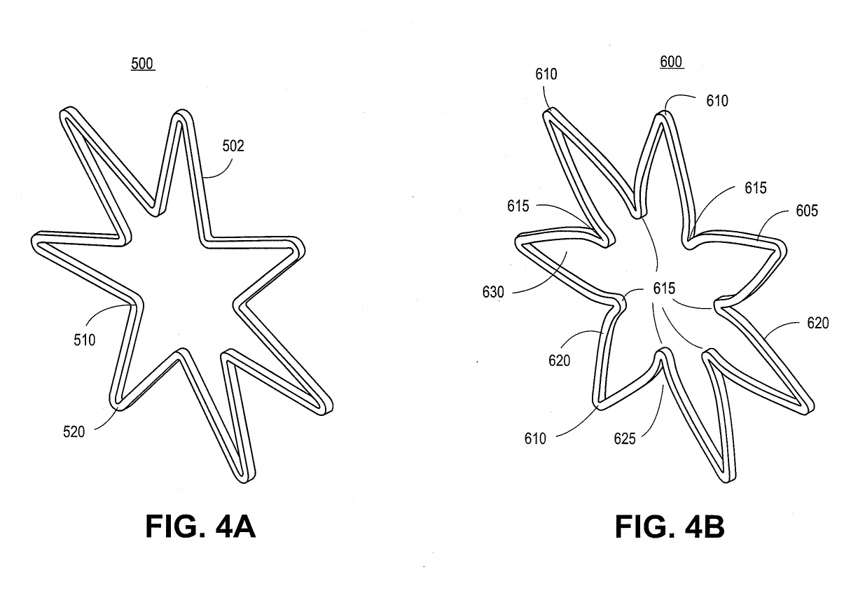

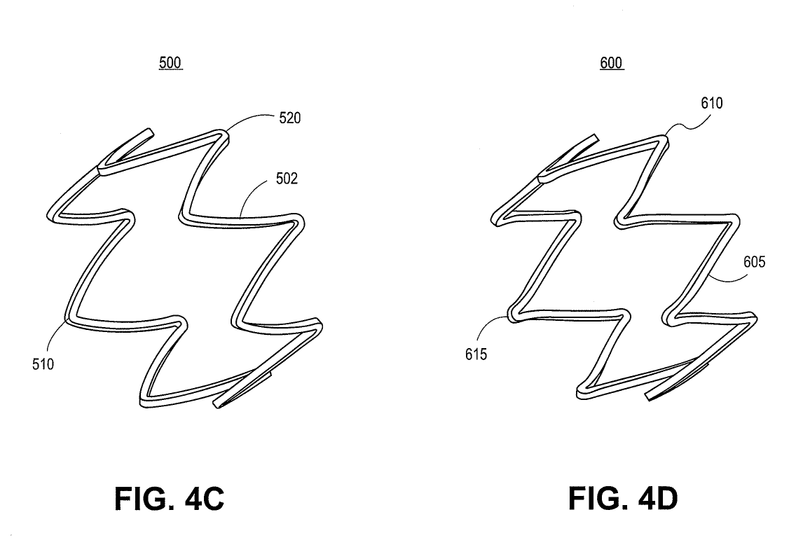

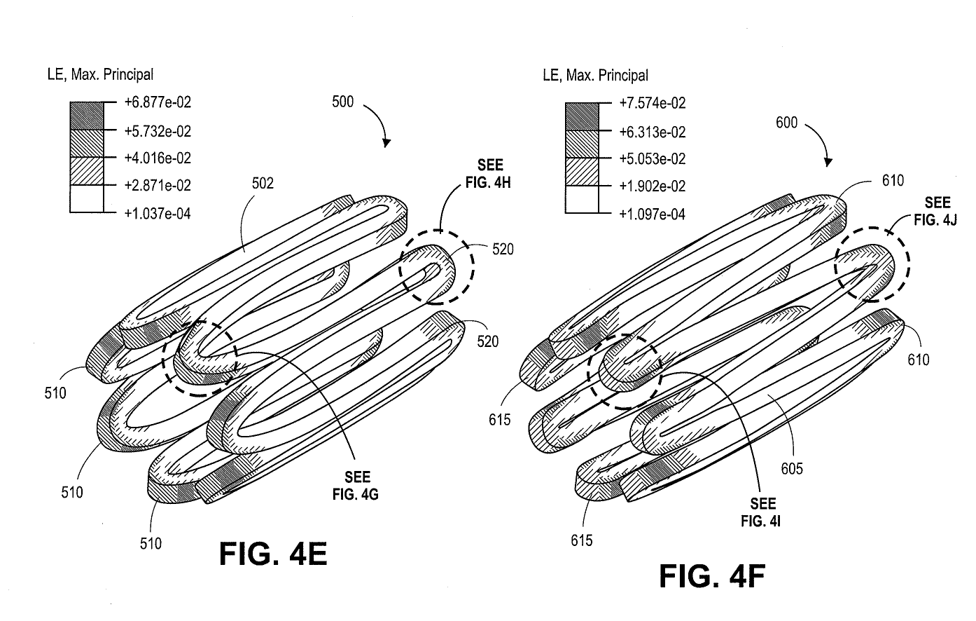

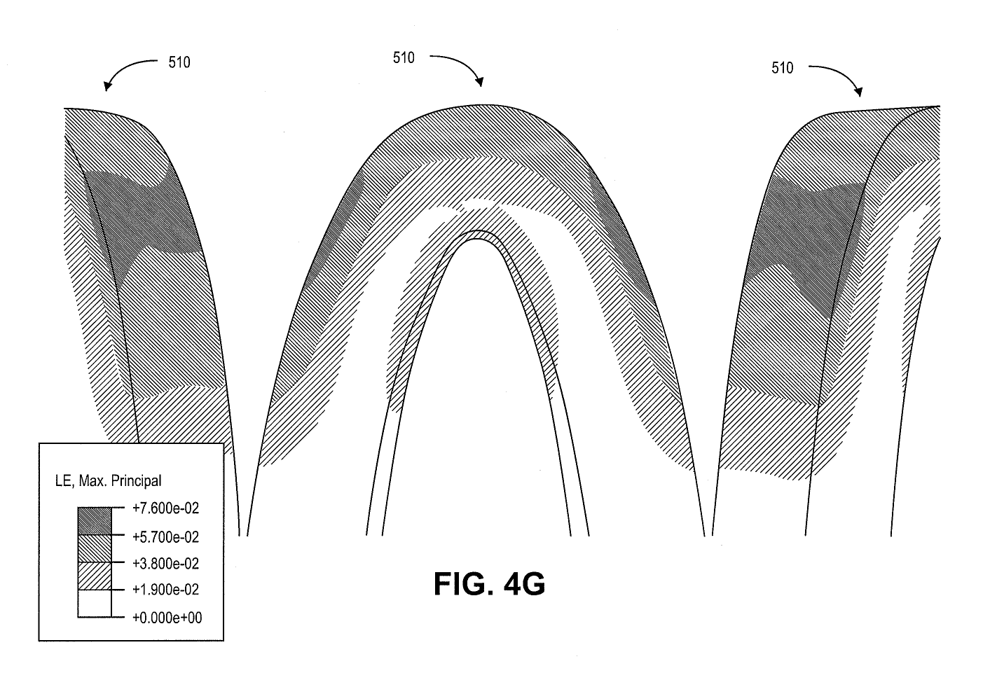

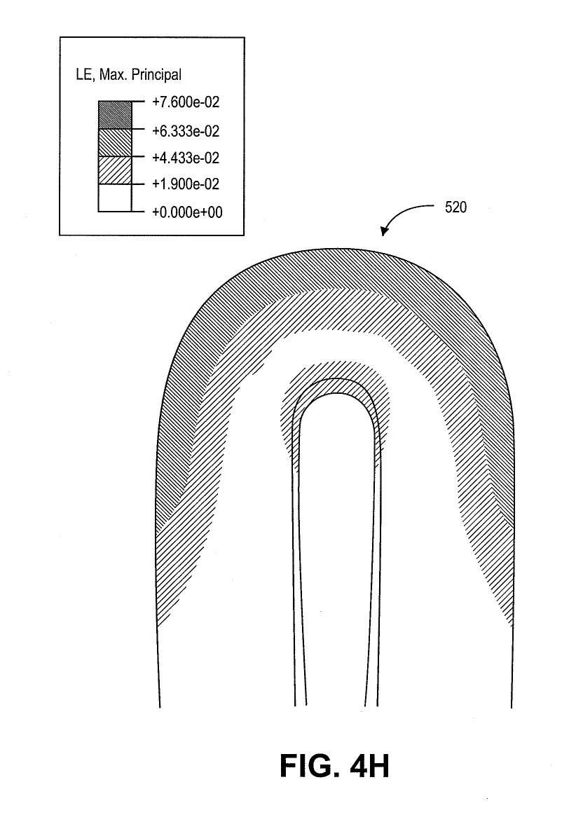

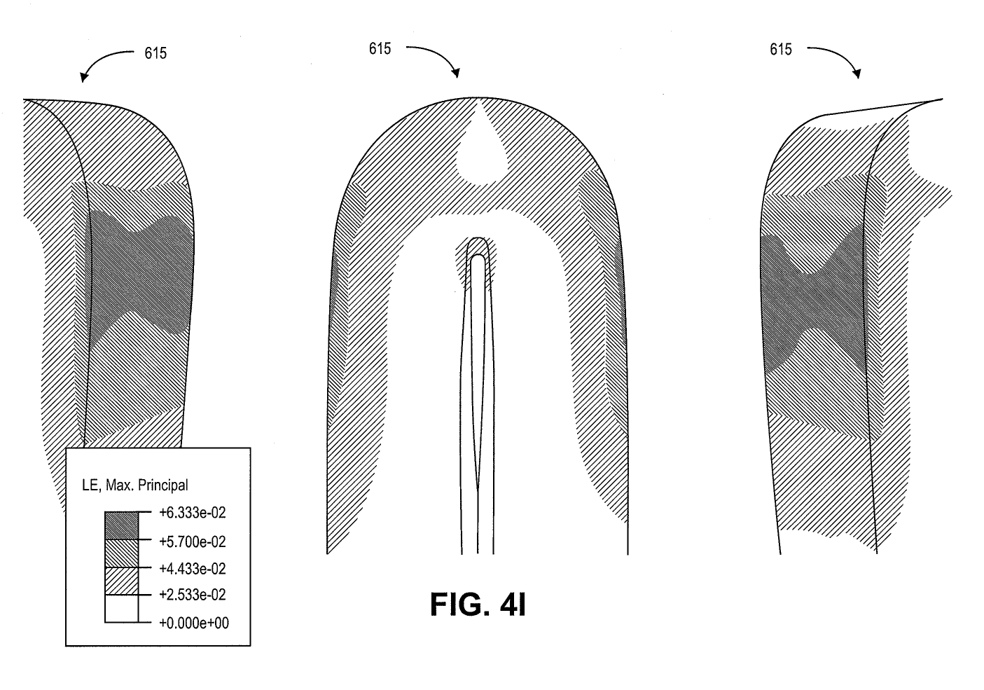

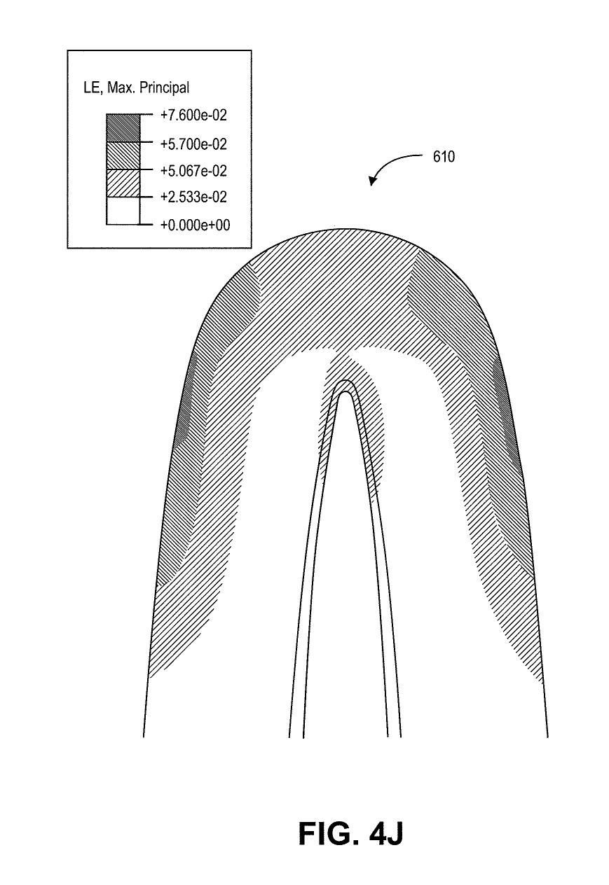

FIG. 3B is an isometric view of the shape of the article 600, when inverted in the manner described in the '893 patent, produces cylindrical parts wherein the two ends are substantially the same (see FIGS. 3B and 4D). Similarly, the angles on the inside of the star shown in FIG. 2A should be less than is shown, and those struts should arch outwards.

In one specific example of an embodiment of the article cut from a planar sheet illustrated in FIG. 3A, the common strut path length distance between an inner apex and an adjacent outer apex is 6.877 mm. In this embodiment, the corresponding straight line distance between the inner apex and the adjacent outer apex is 6.785 mm. When this specific embodiment is everted into the non-planar three dimensional structure illustrated in FIG. 3B, the common strut path length distance between the inner apex and the adjacent outer apex is not appreciably changed. However, the corresponding straight line distance between the inner apex and the adjacent outer apex is increased to 6.876 mm.

FIGS. 4A-F along with the enlarged views of FIGS. 4G-4J compare the crimped strains of parts made per the prior art (4A, 4C, 4E, 4G, and 4H) to those made according to an embodiment the present invention (FIGS. 4B, 4D, 4F, 4I and 4J). In each of the views of FIGS. 4E-4J a graph is provided from the finite element analysis used to simulate the results described herein. In these results, the “LE, max principal” the “LE” references logarithmic strain and “max principal” indicates maximum principal of the strain tensors.

FIGS. 4A and 4B compare the prior art initial geometry with an embodiment of the present invention initial geometry, respectively. The initial geometries are similar to those of the embodiments of FIG. 2A and FIG. 3A, respectively. FIGS. 4C and 4C show both geometries of FIGS. 4A and 4B, respectively, after an eversion process to be formed into a cylindrical shape of 18 mm diameter (Eversion operation performed by finite element analysis). FIGS. 4E and 4F compare the strains after crimping the shapes to 4 mm diameter. Each of FIGS. 4G, 4H, 4I and 4J provide additional comparison of the two structures. A comparison of FIGS. 4E and 4F reveals that the peak strains are reduced from 7.5% (FIG. 4E prior art) to 6.9% (FIG. 4F according to an embodiment of the invention. Further details of the variation between the improved device of FIG. 4F and the convention device of FIG. 4E may appreciated by the enlarged views of the inner and outer apexes in the crimped condition. FIG. 4G is an enlarged view of the inner apexes of the embodiment of FIG. 4E. FIG. 4H is an enlarged view of the outer apexes of the embodiment of FIG. 4E.

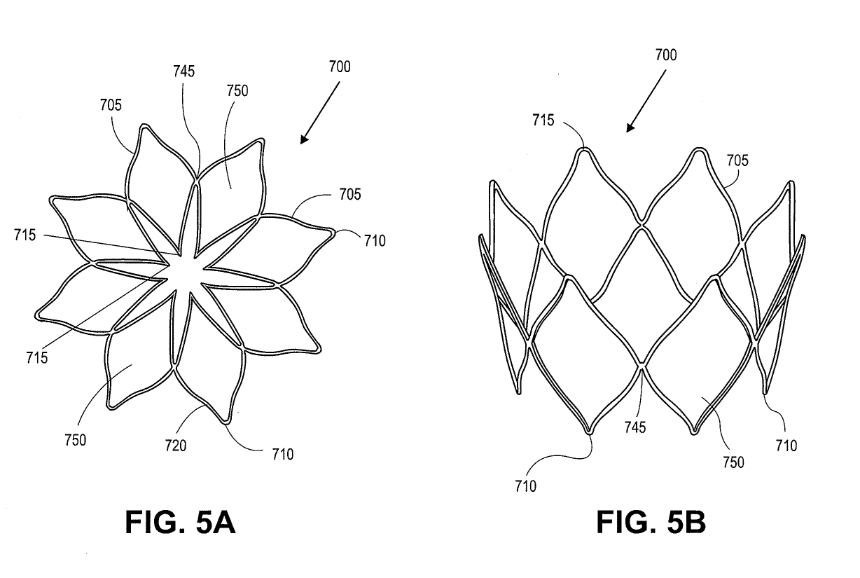

FIGS. 5A and 5B are top down and isometric views, respectively, of a more complex example of a pattern 700 with larger apex angles 710 on the outside than on the inside 715, and the generally planar curvilinear strut configuration (FIG. 5A) before and then after application of the eversion process to form the article into the non-planar three dimensional device illustrated in FIG. 5B.

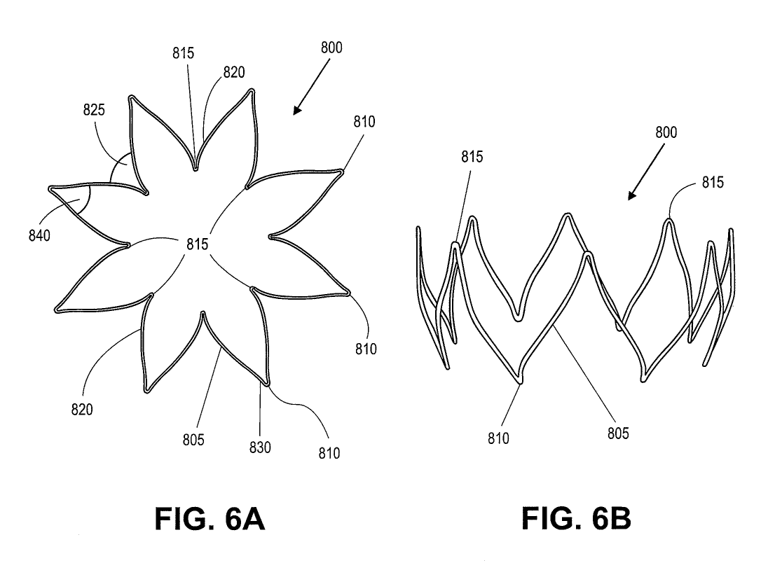

FIGS. 6A and 6B illustrate another pattern 800 and device similar to FIG. 3, The invertible article 800 includes a plurality of curved struts 805 joined together to adjacent struts to form inner apexes 815 and outer apexes 810. Curved struts may have a number of curved sections depending upon the desired design of the final article, i.e., an everted, non-planar three dimensional device. In this illustrative embodiment of FIGS. 6A and 6B, the curved struts 805 include a curved portion 820 near or adjacent to the inner apex 815 and another curved portion 830 near or adjacent to the outer apex 810. The article 800 is set in a semi-conical shape, and with a cross section that is oval rather than circular as the pattern (FIG. 6A) and after the eversion principal, formed into the device 800 shown in FIG. 6B.

We have also found that the tapering issue that exists in prior art devices can be resolved by placing the stents onto tapered mandrel, wherein the part is inverted slightly beyond the desired cylindrical position, and also found that a constraint on the outside of the part is an effective way to assure that the true cylindrical shape is fully achieved (eliminating the outward and inward jutting struts). These curvilinear strut shapes obviate the need for complex fixturing intended to individually bend the struts back to their ideal position.

The method and principles described above apply equally to other, more complicated shapes and to non-cylindrical shapes. FIGS. 5A, 5B, 6A and 6B show two examples of other articles formed according to the improved designs and techniques described herein.

FIG. 5A illustrates a substantially planar article that, everted as in FIG. 5B forms an embodiment of a suprarenal stent 700. As with the previous embodiments, the article includes struts 705 formed into inner apexes 715 and outer apexes 710 with inward and outward angles as before. In addition, the struts of this embodiment include an attachment point 745 between adjacent struts that—when everted—forms the closed shape 750 between each corresponding pair of an inner apex 715 and an outer apex 710. There is a curved portion (720) adjacent to the of a strut adjacent to the outer apex 710.

Returning again to FIGS. 6A and 6B, there is illustrated another embodiment of a substantially planar article 800 formed according to the improvements described above. The article 800 show in FIG. 6A when everted (as shown in FIG. 6B) forms a BIF tapered stent. Also illustrated in the embodiment of the article 800 in FIG. 6A is that each outer apex 810 has at least one strut 805 in common with an adjacent inner apex 815. The struts forming the inward opening angles 840 are convergent and the struts forming the outward opening angles 825 are divergent. In one alternative, the first pattern of struts and the second pattern of struts form a continuous closed shape. In one embodiment, the closed shape is a star pattern, but may include other closed shapes. In still another aspect, the struts forming the inner apexes are curved in a portion 820 of the strut adjacent to the inner apex 815. Struts may also in include curvilinear deigns having a curved section adjacent to an outer apex. A curved section 830 is illustrated adjacent to an outer apex 810 in FIG. 6A. In another aspect, each strut 805 is generally curvilinear along its length between an outer apex and an inner apex. In any of the embodiments described herein, a curvilinear strut design may include curved portions adjacent to an inner apex, or an outer apex, alone or in any combination thereof.

As the embodiment of FIGS. 6A and 6B illustrates, the final shape (i.e., the everted structure) need not be cylindrical but may instead be conical or truncated conical as shown in FIG. 6B.

What is claimed is:

1. An article cut from a metallic sheet, the article comprising:
A first pattern of struts forming a plurality of inner apexes situated substantially within a plane that contains the plurality of the inner apexes, the struts used to form each inner apex providing an outward opening angle; and
A second pattern of struts forming a plurality of outer apexes that are situated substantially within the plane containing the inner apexes, the struts used to form each outer apex providing an inward opening angle, wherein each outer apex has at least one strut in common with an adjacent inner apex, wherein the struts forming the inward opening angle are divergent and the struts forming the outward opening angle are convergent.

2. The article of claim 1 wherein the first pattern of struts and the second pattern of struts form a continuous closed shape.

3. The article of claim 2 wherein the closed shape is a star pattern.

4. The article of claim 1 wherein the struts forming the inner apexes are curved in a portion of the strut immediately adjacent to the inner apex.

5. The article of claim 1 wherein the first pattern and the second pattern are arranged to form an alternating arrangement of an inner apex adjacent to an outer apex.

6. The article of claim 1 wherein the two dimensional article is substantial planar.

7. The article of claim 1 wherein the shape and size of the struts and apexes in the article are selected so that the article forms a non-planar article when everted.

8. The article of claim 7 wherein the non-planar article is substantially cylindrically symmetrical along the longitudinal axis of the non-planar article.

9. The article of claim 7 wherein the non-planar article is a stent adapted and configured for the vasculature of a mammal.

10. The article of claim 1 wherein each strut is generally curvilinear along its length between an outer apex and an inner apex.

11. The article of claim 1 wherein after everting the article forms a device having an approximate conical or cylindrical symmetry.

12. A substantially planar article cut from a metallic sheet for eversion into a non-planar three dimensional structure, the article comprising:
A first pattern of struts forming a plurality of inner apexes;
A second pattern of struts forming a plurality of outer apexes positioned in relation to the first pattern of struts to provide an alternating pattern of an inner apex and an adjacent outer apex;
a common strut that joins the inner apex to the adjacent outer apex wherein a straight line distance between the inner apex and the adjacent outer apex is shorter than a path length along the common strut from the inner apex to the adjacent outer apex.

13. The article of claim 12 wherein after forming the non-planar three dimensional structure, the straight line distance between the inner apex and the adjacent outer apex is longer than in the substantially planar article.

14. The article of claim 12 wherein a curvature of the common strut is greater in the substantially planar article than after conducting an eversion process on the article.

15. The article of claim 12 wherein the plurality of inner apexes define a plurality of outward opening angles and the struts forming the outward opening angle are divergent.

16. The article of claim 12 wherein the plurality of outer apexes define a plurality of inward opening angles and the struts forming the inward opening angle are convergent.

17. The article of claim 12 wherein the angle formed by an inner apex of the substantially planar article is greater than the angle formed by the same inner apex after performing an eversion process.

18. The article of claim 12 wherein the angle formed by an outer apex of the substantially planar article is less than the angle formed by the same outer apex after performing an eversion process.

19. The article of claim 12 wherein each common strut has a first curved portion adjacent to an inner apex and a second different curved portion adjacent to an outer apex.

20. The article of claim 12, the non-planar three dimensional structure further comprising a cylindrical symmetry or a conical symmetry along the main longitudinal axis of the structure.

21. A method of forming a non-planar three dimensional structure, comprising:
patterning a planar sheet of material to form an article having a plurality of inner apexes, a plurality of outer apexes with a common curvilinear strut between an inner apex and an adjacent outer apex wherein the inner apexes form divergent wall included angles and the outer apexes form converging wall included angles; and
everting the article into a non-planar three dimensional structure having the inner apexes at a first end of the structure and the outer apexes at a second end of the structure.

22. The method of claim 21 wherein the size of the inner apexes included angle is reduced after the everting step.

23. The method of claim 21 wherein the side of the outer apexes included angle is increased after the everting step.

24. The method of claim 21 further comprising: performing a heat treatment operation to substantially remove a stress or a strain introduced into the article during the everting step.

25. The method of claim 24 further comprising: reducing the diameter of the non-planar three dimensional structure.

26. The method of claim 21 the everting step further comprising: forming the non-planar three dimensional structure into having a substantially cylindrical symmetry along the main longitudinal axis of the structure.

27. The method of claim 21 the everting step further comprising: forming the non-planar three dimensional structure into having a substantially conical symmetry along the main longitudinal axis of the structure.
